(19) United States
(12) Patent Application Publication
(10) Pub. No.: US 2025/0169796 A1
(43) Pub. Date: May 29, 2025

(54) ULTRASOUND DIAGNOSTIC APPARATUS, ULTRASOUND IMAGE GENERATING METHOD, AND RECORDING MEDIUM

(71) Applicant: Konica Minolta, Inc., Tokyo (JP)

(72) Inventor: Morio NISHIGAKI, Fujisawa-shi (JP)

(21) Appl. No.: 18/955,165

(22) Filed: Nov. 21, 2024

(30) Foreign Application Priority Data
Nov. 28, 2023 (JP) 2023-200491

(51) Int. Cl. A61B 8/08; A61B 8/00; G01S 7/52

(52) U.S. Cl. CPC A61B 8/5207; A61B 8/54; G01S 7/52028

(57) ABSTRACT

An ultrasound diagnostic apparatus includes a hardware processor, and the hardware processor generates image data without saturation from image data with saturation using learned data of a model that has undergone machine learning using the image data with saturation based on a reception signal of an ultrasound probe and the image data without saturation.

### CROSS REFERENCE TO RELATED APPLICATIONS

The present invention claims priority under 35 U.S.C. § 119 to Japanese Application No. 2023-200491, filed Nov. 28, 2023, the entire contents of which being incorporated herein by reference.

### BACKGROUND OF THE INVENTION

### Technical Field

The present invention relates to an ultrasound diagnostic apparatus, an ultrasound image generating method, and a recording medium.

### Description of Related Art

There has been conventionally known an ultrasound diagnostic apparatus that emits ultrasound waves with an ultrasound probe to the interior of a subject, receives the reflected waves, and analyzes the reflected waves to display an ultrasound image of the interior of the subject. The subject is a living body of a patient or the like.

A reception circuit of an ultrasound diagnostic apparatus is required to have a wide dynamic range. Gain setting of the ultrasound diagnostic apparatus is adjusted so that a reception circuit is not saturated. In addition, the gain setting of the ultrasound diagnostic apparatus is adjusted so that a S/N ratio (Signal to Noise Ratio) does not deteriorate because the gain is too low. However, the reception signal greater than expected may be input to the reception circuit. At this time, the reception circuit may be saturated. In this case, for example, in a tomographic image, image quality deterioration such as flow in the horizontal direction occurs.

Here, conventional gain adjustment of the reception signal is described with reference to FIG. 7. FIG. 7 is a block diagram illustrating a transducer 211 of a conventional ultrasound probe and a receiver 33. The receiver 33 includes a pre-amplifier 331, a variable gain amplifier 332, an analog-to-digital (AD) converter 333, and a beam former 334. The transducer 211 includes transducers 211a to 211h. The pre-amplifier 331 includes pre-amplifiers 331a to 331h. The variable gain amplifier 332 includes variable gain amplifiers 332a to 332h. The AD converter 333 includes AD converters 333a to 333h.

In FIG. 7, a transmitter that transmits a drive signal to the transducers 211a to 211h of the ultrasound probe and a configuration that generates image data from sound ray data and displays it on a display are omitted from illustration. In addition, the transducers 211a to 211h and portions corresponding thereto representatively illustrate the transducers of the ultrasound probe, and the number thereof is not limited to the number shown here (eight).

Ultrasound waves emitted from the transducers 211a to 211h of the ultrasound probe are reflected off a subject, received as reflected ultrasound waves (echoes) by the transducers 211a to 211h, and converted into reception signals. The reception signal is an electrical signal.

The reception signals output from the transducers 211a to 211h are amplified by the pre-amplifiers 331a to 331h and further amplified by the variable gain amplifiers 332a to 332h. The amplified reception signals are converted into digital signals by the AD converters 333a to 333h, and the digital signals are subjected to phasing addition by the beam former 334 to generate sound ray data.

Here, the gain control is described. The ultrasound waves received by the transducers 211a to 211h have a wide dynamic range. In contrast, the dynamic ranges of the pre-amplifiers 331a to 331h, the variable gain amplifiers 332a to 332h, the AD converters 333a to 333h, and the beam former 334 after conversion into reception signals are narrow. In particular, the dynamic range of the AD converters 333a to 333h and subsequent components is narrow. Therefore, amplification factors of the variable gain amplifiers 332a to 332h are adjusted so that the AD-converted digital signals have appropriate amplitudes.

Generally, the closer to a body surface of the subject, the shorter a propagation distance is, so that the amplitude of the reception signal is large. The amplitude of the reception signal decreases as a depth from the body surface increases. However, depending on a shape of a tissue interface and a magnitude of an impedance difference, the amplitude of the reception signal will vary. For this reason, when a reception signal larger than expected enters from the transducers 211a to 211h, saturation occurs in a circuit in a subsequent stage.

The saturation may occur due to the ultrasound wave input to all of the transducers 211 or due to the ultrasound wave input to some of the transducers. However, it is difficult to perform optimum gain control depending on the subject, the part to which the ultrasound probe is applied, and the way in which the ultrasound probe is applied.

When the saturation occurs, for example, an error may occur in phasing addition in the beam former 134, and a correct beam may not be formed. Furthermore, in an image mode in which a blood flow signal is displayed, a small-amplitude blood flow echo is superimposed on a large-amplitude body tissue echo. Therefore, when the body tissue echo is saturated, the blood flow echo may disappear. In addition, when the gain is excessively lowered so as not to be saturated, the signal is buried in noise, and thus it is not possible to perform extraction.

Generally, the adjustment of the amplifier of the receiver is not included in adjustment items by the user. Gain adjustment of the ultrasound diagnostic apparatus includes comprehensive gain adjustment and TCG (Time Control Gain) adjustment. However, these are not adjustments of the amplifier of the receiver. Therefore, when the ultrasound image is saturated, adjustment for eliminating the saturation cannot be performed in many cases. In addition, it is difficult for the user to perform appropriate adjustment. Furthermore, it is necessary to increase power consumption in order to secure a sufficient dynamic range. In particular, in recent portable ultrasound diagnostic apparatuses, saturation is likely to occur because power consumption is suppressed. The deterioration of the image quality of the ultrasound image in the case of saturation is considerably conspicuous. From the viewpoint of quality, it is necessary to take measures to prevent saturation or to prevent deterioration of the image even when saturation occurs.

Therefore, an ultrasound diagnostic apparatus is known that determines saturation in accordance with odd-order harmonic components extracted from reception signals (reflected wave signals) received by the elements of an ultrasound probe (see Japanese Unexamined Patent Publication No. 2023-109051). The ultrasound diagnostic apparatus multiplies the reception signal determined to be saturated by a weight coefficient (decreases the gain) and generates sound ray data (reflected wave data) from the multiplied reception signal.

In addition, an ultrasound diagnostic apparatus having a saturation estimation function that performs saturation estimation of a reflected wave signal that has passed through an ultrasound probe and a transmission/reception circuit is known (see Japanese Unexamined Patent Publication No. 2019-97794). When even one of the reflected wave signals has an amplitude exceeding a threshold value, the saturation estimation function sets all the packet data to 0. This ultrasound diagnostic apparatus interpolates (fills) moving object information of an observation point at which packet data is missing from surrounding moving object information.

In addition, an ultrasound diagnostic apparatus having a learned model that generates output data based on a high sound pressure signal of an ultrasound wave by using input data based on a low sound pressure signal of an ultrasound wave is known (see Japanese Patent No. 7273519). The ultrasound diagnostic apparatus generates, using the learned model, output data based on the high sound pressure signal by inputting input data based on the low sound pressure signal of the ultrasound wave acquired by the inspection.

The ultrasound diagnostic apparatus of Japanese Unexamined Patent Publication No. 2023-109051 reduces the effect of signal degradation on saturation. However, on the contrary, the amplitude balance between the elements (channels) is lost. For this reason, a problem may occur in the beam shape. Further, the ultrasound diagnostic apparatus of Japanese Unexamined Patent Publication No. 2019-97794 interpolates a saturated observation point with surrounding moving object information. Therefore, better image quality is required.

Further, the ultrasound diagnostic apparatus of Patent Document 7273519 does not reduce the influence of signal deterioration in saturation.

### SUMMARY OF THE INVENTION

An object of the present invention is to obtain a high-quality ultrasound image without saturation even in the case of saturation due to a strong echo.

To achieve at least one of the abovementioned objects, according to an aspect of the present invention, an ultrasound diagnostic apparatus reflecting one aspect of the present invention includes, a hardware processor, wherein the hardware processor generates image data without saturation from image data with saturation using learned data of a model that has undergone machine learning using the image data with saturation based on a reception signal of an ultrasound probe and the image data without saturation.

According to another aspect of the present invention, an ultrasound image generating method includes: generating image data without saturation from image data with saturation using learned data of a model that has undergone machine learning using the image data with saturation based on a reception signal of an ultrasound probe and the image data without saturation.

According to another aspect of the present invention, a non-transitory computer-readable recording medium stores a program that causes a computer to: as a controller, generate image data without saturation from image data with saturation using learned data of a model that has undergone machine learning using the image data with saturation based on a reception signal of an ultrasound probe and the image data without saturation.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic diagram of an ultrasound diagnostic apparatus according to a first embodiment of the present invention.

FIG. 2 is a block diagram illustrating a functional configuration of the ultrasound diagnostic apparatus.

FIG. 3 is a block diagram of the ultrasound diagnostic apparatus illustrating an internal configuration of a receiver.

FIG. 4 is a flowchart illustrating learning processing.

FIG. 5 is a flowchart illustrating the first image display processing.

FIG. 6 is a flowchart illustrating second image display processing.

FIG. 7 is a block diagram illustrating a transducer and a receiver of a conventional ultrasound probe.

### DETAILED DESCRIPTION

Hereinafter, one or more embodiments of the present invention will be described with reference to the drawings. However, the scope of the invention is not limited to the disclosed embodiments.

Hereinafter, first and second embodiments of the present invention will be described in order with reference to the accompanying drawings. However, the scope of the invention is not limited to the illustrated examples.

### First Embodiment

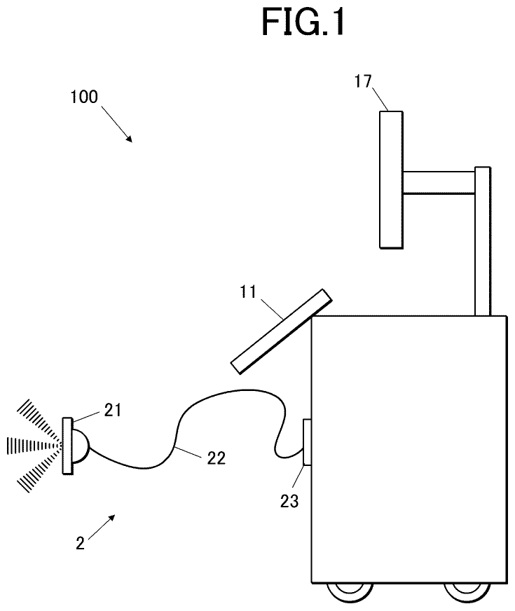

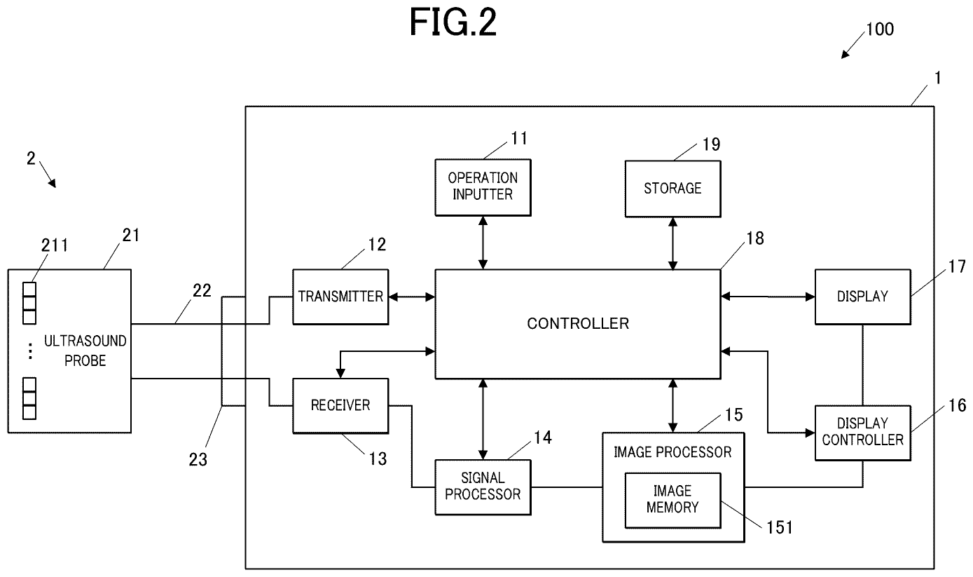

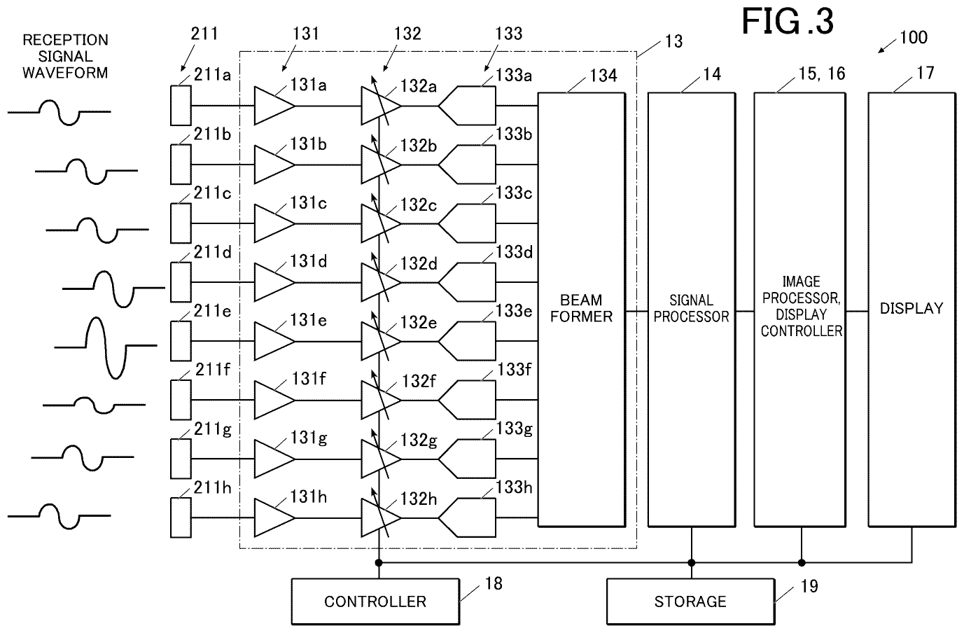

A first embodiment of the present invention will be described with reference to FIG. 1 to FIG. 5. First, the apparatus configuration of the present embodiment will be described with reference to FIG. 1 to FIG. 5. FIG. 1 is a schematic view of an ultrasound diagnostic apparatus 100 according to the present embodiment. FIG. 2 is a block diagram illustrating a functional configuration of the ultrasound diagnostic apparatus 100. FIG. 3 is a block diagram of the ultrasound diagnostic apparatus 100 illustrating an internal configuration of the receiver 13.

As illustrated in FIG. 1, the ultrasound diagnostic apparatus 100 is provided in a medical facility such as a hospital and generates ultrasound image data by emitting ultrasound waves to a subject such as a living body of a patient. The ultrasound diagnostic apparatus 100 also estimates ultrasound image data by using a learned model of machine learning. The ultrasound diagnostic apparatus 100 is configured to display a color Doppler image in a color Doppler mode. The color Doppler image in the color Doppler mode is a display image of ultrasound image data in which color Doppler (color flow) image data and brightness (B) image data are superimposed. The color Doppler image data is ultrasound image data of a tomographic image indicating a blood flow state of the subject in color. The B image data is the ultrasound image data of the tomographic image in which a tissue or the like of the subject is indicated by luminance.

The ultrasound diagnostic apparatus 100 includes an ultrasound diagnostic apparatus main body 1 and an ultrasound probe 2. The ultrasound probe 2 is connected to the ultrasound diagnostic apparatus main body 1. The ultrasound probe 2 transmits ultrasound waves (transmitted ultrasound waves) into a subject and receives ultrasound waves reflected inside the subject (reflected ultrasound waves: echoes). The ultrasound probe 2 has an ultrasound probe main body 21, a cable 22, and a connector 23. The ultrasound probe main body 21 is a header of the ultrasound probe 2 and transmits and receives ultrasound waves. The cable 22 is connected to the ultrasound probe main body 21 and the connector 23. The cable 22 is a cable through which a drive signal for the ultrasound probe main body 21 and a reception signal of ultrasound waves flow. The connector 23 is a plug connector for establishing a connection with a receptacle connector (not illustrated) of the ultrasound diagnostic apparatus main body 1.

The ultrasound diagnostic apparatus main body 1 is connected to the ultrasound probe main body 21 via the connector 23 and the cable 22. The ultrasound diagnostic apparatus main body 1 transmits a drive signal, which is an electric signal, to the ultrasound probe main body 21 to direct the ultrasound probe main body 21 to transmit transmission ultrasound waves to the subject. The ultrasound probe 2 generates a reception signal, which is an electric signal, according to the reflected ultrasound waves from the inside of the subject received by the ultrasound probe main body 21. The ultrasound diagnostic apparatus main body 1 images the internal state of the subject as ultrasound image data on the basis of the reception signal generated by the ultrasound probe 2.

The ultrasound probe main body 21 includes a transducer 211 (FIG. 2) on a distal end side. The transducer 211 includes transducers 211a to 211h (FIG. 3). The eight transducers 211a to 211h are the representatives of the transducers 211 for ease of description. The number of transducers 211 can be set as desired. The actual number of transducers is 192, for example.

Multiple transducers 211 are arranged, for example, in a one-dimensional array in a scanning direction (azimuth direction). The transducers 211 may be arranged in a two-dimensional array. In the present embodiment, the ultrasound probe 2 is a linear scanning type electronic scanning probe. However, the ultrasound probe 2 may be of either an electronic scanning type or a mechanical scanning type. In addition, the ultrasound probe 2 may be of any of a linear scanning type, a sector scanning type, and a convex scanning type. The ultrasound diagnostic apparatus main body 1 and the ultrasound probe 2 may be configured to perform wireless communication between each other instead of wired communication via the cable 22. The wireless communication is an ultra-wide band (UWB), for example.

The operation inputter 11 is a control panel or the like configured to receive various operation inputs by a user, such as a doctor or a technician. The operation inputter 11 includes operational elements, such as a push button, an encoder, a lever switch, a joystick, a trackball, a keyboard, a touch pad, and/or a multifunction switch.

The display 17 includes a display panel such as a liquid crystal display (LCD), an organic electro-luminescence (EL) display, or an inorganic EL display. The display 17 displays display information such as ultrasound image data on the display panel.

As illustrated in FIG. 2, the ultrasound diagnostic apparatus main body 1 includes an operation inputter 11, a transmitter 12, a receiver 13, a signal processor 14, an image processor 15, a display controller 16, a display 17, a controller 18 (hardware processor), and a storage 19.

The operation inputter 11 receives various operation inputs from the user, and outputs the operation signals to the controller 18. The operation inputter 11 may include a touch screen formed integrally with the display screen of the display 17 and configured to receive touch inputs by the user. Furthermore, the operation inputter 11 receives an operation input of a display mode of blood flow components (blood flow velocity, power, and dispersion) in the color Doppler mode. The operation inputter 11 also receives an operation input of a region of interest (ROI) for a color Doppler image.

Under the control of the controller 18, the transmitter 12 supplies a drive signal, which is an electric signal, to the ultrasound probe 2 to direct the ultrasound probe 2 to generate transmission ultrasound waves. The transmitter 12 includes, for example, a clock generation circuit, a delay circuit, and a pulse generation circuit. The clock generation circuit generates a clock signal for determining the transmission timing and transmission frequency of the drive signal. The delay circuit sets a delay time for each individual path corresponding to each transducer 211 and delays the transmission of the drive signal by the set delay time. The delay circuit focuses transmission beams formed by the transmission ultrasound waves by the delay. The pulse generating circuit generates a pulse signal as a drive signal at a predetermined cycle. The transmitter 12 drives, for example, a consecutive part (e.g., 64) of the plurality of (e.g., 192) transducers 211 arrayed in the ultrasound probe 2 to generate transmission ultrasound waves. Then, the transmitter 12 performs scanning by shifting the driven transducer 211 in the scanning direction each time the transmission ultrasound is generated.

Further, the transmitter 12 generates a drive signal corresponding to the ROI input by the operation inputter 11 for the color Doppler image data under the control of the controller 18. The drive signal corresponding to the ROI of the color Doppler image is the drive signal for transmitting a plurality of ultrasound waves to the same scanning line. In addition, the transmitter 12 generates a drive signal corresponding to the entire region for brightness (B) mode image data under the control of the controller 18.

The receiver 13 receives a reception signal, which is an electric signal, from the ultrasound probe 2 under the control of the controller 18. Here, an internal configuration of the receiver 13 will be described with reference to FIG. 3. The receiver 13 includes a pre-amplifier 131, a variable gain amplifier 132, an AD converter 133, and a beam former 134. The pre-amplifier 131 includes pre-amplifiers 131a to 131h. The variable gain amplifier 132 includes variable gain amplifiers 132a to 132h. The AD converter 133 includes AD converters 133a to 133h. The number of each of the pre-amplifiers 131, the variable gain amplifiers 132, and the AD converters 133 is the number (eight) corresponding to the transducers 211a to 211h but is not limited thereto.

The pre-amplifiers 131a to 131h amplify the voltages of the reception signals generated by the transducers 211a to 211h by predetermined gain values (amplification factors) set in advance. Under the control of the controller 18, the variable gain amplifiers 132a to 132h amplify the voltages of the reception signals amplified by the pre-amplifiers 131a to 131h by any gain values (amplification factors). The AD converters 133a to 133h convert the analog reception signals amplified by the variable gain amplifiers 132a to 132h into digital reception signals.

The beam former 134 adjusts the time phases of the reception signals A/D-converted by the AD converters 133a to 133h by providing them with delay times for the individual paths corresponding to the respective transducers 211. The beam former 134 generates sound ray data by adding (phasing addition) the reception signals after these processes.

When generating B-mode image data, the signal processor 14 performs envelope detection processing, logarithmic compression, and the like on the sound ray data from the receiver 13 under the control of the controller 18. After the above processes, the signal processor 14 further adjusts the dynamic range and the gain of the sound ray data to convert the data into brightness. The signal processor 14 generates B-mode image data consisting of pixels having a luminance value as received energy, by the luminance conversion. That is, the B-mode image data represents the intensity of the reception signals by brightness.

Further, the signal processor 14 generates the color Doppler image data. In this case, the signal processor 14 generates the color Doppler image data of the ROI in accordance with the sound ray data input from the receiver 13 under the control of the controller 18. This ROI is an ROI input via the operation inputter 11. The signal processor 14 includes, for the color Doppler mode, a quadrature detection circuit, a corner turn controller, a moving target indicator (MTI) filter, a correlation calculator, a data converter, a noise removal spatial filter, an inter-frame filter, and a color Doppler image converter.

Under the control of the controller 18, the quadrature detection circuit performs quadrature detection on the reception signals in the color Doppler mode input from the receiver 13. The quadrature detection circuit calculates a phase difference between the acquired reception signal in the color Doppler mode and a reference signal by quadrature detection, and acquires (complex) Doppler signals I and Q. The corner turn controller arranges the Doppler signals I and Q input from the quadrature detection circuit under the control of the controller 18. The array is an array of a depth direction from the ultrasound probe to the subject and an ensemble direction of the number n of repetitions of transmission and reception of ultrasound waves for each identical acoustic line (line). The corner turn controller stores the arrayed Doppler signals I and Q in a memory (not shown) and reads the Doppler signals I and Q for each depth in the ensemble direction. The reception signals (Doppler signals I and Q) include not only signal components of a blood flow necessary for generating a color flow image but also information (clutter components) of an unnecessary blood vessel wall, tissue, and the like. Under the control of the controller 18, the MTI filter filters the Doppler signals I and Q input from the corner turn controller to remove clutter components.

The correlation calculator calculates a real part D and an imaginary part N of an average value S of the autocorrelation calculation of the Doppler signal from the Doppler signals I and Q from the MTI filter under the control of the controller 18. The Doppler signals I and Q are complex Doppler signals z. The average value S of the autocorrelation calculation of the Doppler signal is an average value of the phase difference vectors. Under the control of the controller 18, the data converter calculates the blood flow component from the Doppler signals I and Q from the MTI filter and the real part D and the imaginary part N of the average value S of the autocorrelation calculation. The blood flow components are blood flow velocity, power, and dispersion.

The noise removal spatial filter filters, under the control of the controller 18, the power, the blood flow velocity, and the dispersion calculated by the data converter. The inter-frame filter performs inter-frame filtering of the blood flow component from the noise removal spatial filter under the control of the controller 18. The inter-frame filter selects a blood flow component forming the color Doppler image from the noise removal spatial filter in accordance with the display mode of the color Doppler mode from the operation inputter 11. The inter-frame filter performs filtering so as to smooth a change between frames of the selected blood flow component and leave an afterimage. Under the control of the controller 18, the color Doppler image convertor color-maps the blood flow component from the inter-frame filter and converts and generates it into color flow image data of the ROI. For example, in the color Doppler image data corresponding to the blood flow velocity, a blood flow flowing in a direction toward the ultrasound probe 2 is expressed in red. In the color Doppler image data, a blood flow that similarly flows in a direction away is expressed in blue.

The image processor 15 includes an image memory 15a. The image memory 15a is constituted of a semi-conductor memory, such as a dynamic random access memory (DRAM), for example. Under the control of the controller 18, the image processor 15 stores the B-mode image and the color Doppler image transmitted from the signal processor 14 in the image memory 15a in units of frames. The image processor 15 transmits the B-mode image and the color Doppler image stored in the image memory 15a to the display controller 16 on a frame-by-frame basis at predetermined time intervals.

The display controller 16 is, for example, a digital scan converter (DSC). Under the control of the controller 18, the display controller 16 performs processing, such as coordinate conversion, on the ultrasound image data received from the image processor 15, to convert the data into image signals for display. In particular, the display controller 16 performs processing for superimposing the color Doppler image data and the B-mode image data in the color Doppler mode. The display controller 16 outputs the image signals to the display 17.

Under the control of the controller 18, the display 17 displays an ultrasound image on the display panel in accordance with the image signal outputted from the display controller 16. Furthermore, the display 17 displays various display information inputted from the controller 18 on the display panel.

The controller 18 includes, for example, a central processing unit (CPU), a read only memory (ROM), and a random access memory (RAM). The controller 18 reads various processing programs stored in the ROM, develops them in the RAM, and controls the units of the ultrasound diagnostic apparatus 100 by cooperation between the developed programs and the CPU. The ROM includes a nonvolatile memory such as a semiconductor. The ROM stores a system program corresponding to the ultrasound diagnostic apparatus 100, various processing programs executable on the system program, various data such as a gamma table, and the like. In particular, the ROM stores a learning program for executing learning processing, which will be described later, and a first image display program for executing first image display processing, which will be described later. These programs are stored in the RAM in the form of computer-readable program codes. The CPU sequentially executes operations according to the program codes on the RAM. The RAM forms a work area in which various programs executed by the CPU and data related to these programs are temporarily stored.

The storage 19 is a storage unit such as a hard disk drive (HDD), a solid state drive (SSD) or the like that stores information such as ultrasound image data in a writable and readable manner. In particular, the storage 19 stores learned data as a learned model of machine learning. The learned data includes determination data and estimation data. The determination data is data for determining the presence or absence of a saturated region at any position in the ultrasound image data. The estimation data is data for estimating and generating ultrasound image data of a non-saturated region from ultrasound image data of a saturated region.

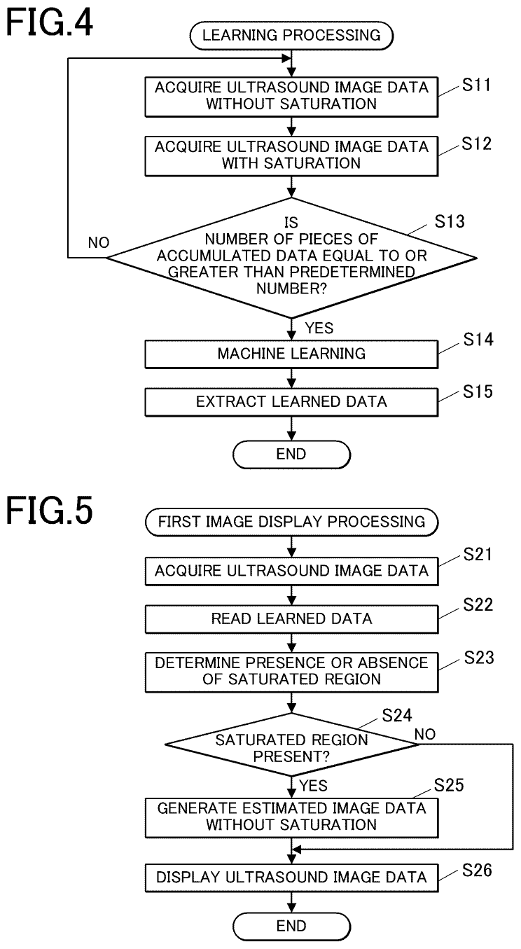

Next, the operation of the ultrasound diagnostic apparatus 100 according to the present embodiment will be described with reference to FIG. 4 and FIG. 5. FIG. 4 is a flowchart illustrating learning processing. FIG. 5 is a flowchart illustrating first image display processing.

First, learning processing executed by the ultrasound diagnostic apparatus 100 will be described with reference to FIG. 4. The learning processing is processing of acquiring, as teacher data, ultrasound image data with/without saturation for a subject such as a patient serving as a learning sample and performing machine learning.

In the ultrasound diagnostic apparatus 100, for example, an instruction to execute learning processing is input from the user via the operation inputter 11. In response to the execution instruction, the controller 18 executes the learning process in accordance with the learning program stored in the ROM.

First, the controller 18 acquires an ultrasound image without saturation and stores it in the storage 19 under the control of the transmitter 12 to the display controller 16 (step S11). The non-saturated ultrasound image data of step S11 is color Doppler image data. The ultrasound image data without saturation is obtained by, for example, controlling the gain values of the variable gain amplifiers 132a to 132h to be low.

Under the control of the transmitter 12 to the display controller 16, the controller 18 acquires the saturated (having a saturated region) ultrasound image and stores it in the storage 19 (step S12). The ultrasound image data with saturation in step S12 is also color Doppler image data. The saturated ultrasound image data can be obtained by, for example, controlling the gain values of the variable gain amplifiers 132a to 132h to be high. Provided that the ultrasound image data with/without saturation may be generated in advance by the own apparatus or another ultrasound diagnostic apparatus and stored in the storage 19. With this configuration, in steps S11 and S12, the controller 18 reads and acquires the ultrasound image data with/without saturation from the storage 19.

In addition, a configuration may be adopted in which ultrasound image data with/without saturation captured in advance by the own apparatus or another ultrasound diagnostic apparatus is stored in a device such as a server. In this configuration, in steps S11 and S12, for example, the controller 18 receives and acquires the ultrasound image data with/without saturation from the device via the communicator (not shown).

The controller 18 determines whether the number of accumulated ultrasound image datasets with/without saturation stored in the storage 19 is equal to or more than a predetermined number (step S13). The predetermined number in step S13 is the number of accumulated data sufficient for machine learning of ultrasound image data with/without saturation. The machine learning estimates the boundaries of feature amounts using, as teacher data, ultrasound image data with/without saturation accumulated in the storage 19, for example. Furthermore, the machine learning generates, as learned data, data for determination of ultrasound image data with/without saturation using the boundary. Furthermore, the machine learning generates, as learned data, estimation data for estimating the non-saturated ultrasound image data from the saturated ultrasound image data using the boundary.

When the number is less than the predetermined number (step S13; NO), the process proceeds to step S11. If the number is equal to or larger than the predetermined number (step S13; YES), the controller 18 performs machine learning using the ultrasound image data with/without saturation in the storage 19 (step S14). The controller 18 extracts learned data from the learning result of the machine learning in step S14 and stores the learned data in the storage 19 (step S15). The learning processing ends. The learned data serves as determination data and estimation data of the color Doppler image data.

Next, the first image display processing executed by the ultrasound diagnostic apparatus 100 will be described with reference to FIG. 5. The first image display processing is processing to acquire ultrasound image data of a subject such as a patient to be diagnosed and, if there is a saturated region, estimate, generate, and display estimated image data without saturation.

In the ultrasound diagnostic apparatus 100, after the learning processing, for example, an instruction to execute the first image display processing is input from the user via the operation inputter 11. In response to the execution instruction, the controller 18 executes the first image display processing in accordance with the first image display program stored in the ROM.

First, under the control of the transmitter 12 to the display controller 16, the controller 18 acquires ultrasound image data of a subject as a target to be diagnosed (step S21). The ultrasound image data in step S21 includes the color Doppler image data and the B-mode image data to be superimposed thereon. The controller 18 reads the learned data from the storage 19 (step S22). The controller 18 determines, using the determination data of the learned data of step S22, whether or not the ultrasound image data of step S21 includes the saturated region (step S23). In step S23, the color Doppler image data in step S21 is determined. The controller 18 determines, from the determination result of step S23, whether or not there is a saturated region (step S24).

If there is a saturated region (step S24; YES), the process proceeds to step S25. The controller 18 estimates and generates, from the ultrasound image data of the region with saturation determined in step S24, ultrasound image data of the same position region without saturation (step S25). The determination data of the learned data in step S22 is used for the estimation and generation. In step S25, the controller 18 generates the ultrasound image data in which the saturated region is replaced with the non-saturated region. The ultrasound image data including the estimated and generated non-saturated region is set as estimated image data. In step S25, the estimated image data becomes the color Doppler image data without saturation.

After execution of step S25, or when it is not saturated (step S24; NO), the process proceeds to step S26. The controller 18 causes the display 17 to display the ultrasound image data of step S21 or the estimated image data of step S25 (step S26). When step S25 is not executed, the controller 18 superimposes and displays the color Doppler image data and the B-mode image data in step S21. When step S25 is executed, the controller 18 displays the color Doppler image data, which is the estimated image data in step S25, and the B-mode image data in step S21 in a superimposed manner. The first image display processing ends.

It is preferable that the image generation conditions of the ultrasound image with/without saturation in the learning processing correspond to the image generation conditions of the ultrasound image in step S21 of the first image display processing. The image generation conditions include various processing conditions related to generation of ultrasound image data, such as transmission conditions, reception conditions, an image mode, and image processing. The image generation conditions may include the type and part of the subject.

In the present embodiment, the saturation is not observed in the individual transducers 211a to 211h (channels), but the saturation is determined (detected) from the image data after the phasing addition. Furthermore, in the present embodiment, machine learning is performed on the basis of saturated ultrasound image data (having a region) and unsaturated ultrasound image data. The non-saturated ultrasound image data is estimated and generated from the ultrasound image data including the saturated region using the estimation data of the learned data. Thus, the image quality of the ultrasound image data (color Doppler image data) is improved. A configuration may be adopted in which machine learning is performed based on the ultrasound image data with saturation of the entire region and the ultrasound image data without saturation. With this configuration, the non-saturated ultrasound image data is estimated and generated from the saturated ultrasound image data of the entire region using the estimation data of the learned data.

According to the present embodiment, the ultrasound diagnostic apparatus 100 includes the controller 18. The controller 18 generates learned data of a learned model obtained by machine learning using the ultrasound image data with saturation based on the reception signals of the ultrasound probe 2 and the ultrasound image data without saturation. The controller 18 generates the ultrasound image data without saturation from the ultrasound image data with saturation using the learned data. In addition, the controller 18 performs machine learning using the ultrasound image data with saturation based on the reception signal of the ultrasound probe 2 and the ultrasound image data without saturation. The controller 18 generates learned data of a learned model of the machine learning.

Therefore, even in the case of saturation due to a strong echo, it is possible to obtain an ultrasound image (color Doppler image) with good image quality without saturation. In particular, in color Doppler image data, a blood flow echo can be prevented from disappearing.

The controller 18 determines, with the determination data of the learned data, whether the ultrasound image data based on the reception signals of the ultrasound probe 2 is saturated. The learned data includes determination data for determining whether the ultrasound image data is saturated. When saturation exists, the controller 18 estimates and generates ultrasound image data without saturation from the ultrasound image data by the estimation data of the learned data. The learned data includes the estimation data for generating non-saturated ultrasound image data from saturated ultrasound image data. Therefore, whether or not the ultrasound image data is saturated can be more accurately determined, and even in a case of saturation due to a strong echo, the ultrasound image without saturation and with better image quality can be obtained.

The controller 18 determines whether or not the ultrasonic data based on the reception signal of the ultrasound probe 2 includes a saturated region by the determination data. The determination data is data for determining whether the ultrasound image data includes the saturated region. When there is the saturated region, the controller 18 estimates and generates the ultrasound image data of the non-saturated region from the ultrasound image data of the saturated region using the estimation data. The controller 18 generates the ultrasound image data including the non-saturated region. The estimation data is data for estimating and generating the ultrasound image data of the non-saturated region from the ultrasound image data of the saturated region and generating the ultrasound image data including the non-saturated region. Therefore, it is possible to more accurately determine whether the ultrasound image data includes the saturated region. In addition, even in a case of saturation due to strong echo, the ultrasound image without saturation and with better image quality can be obtained, and the image processing burden can be reduced.

### Second Embodiment

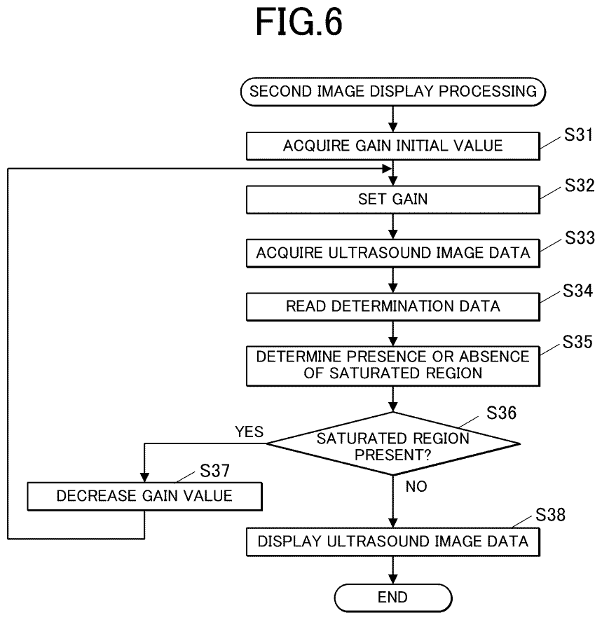

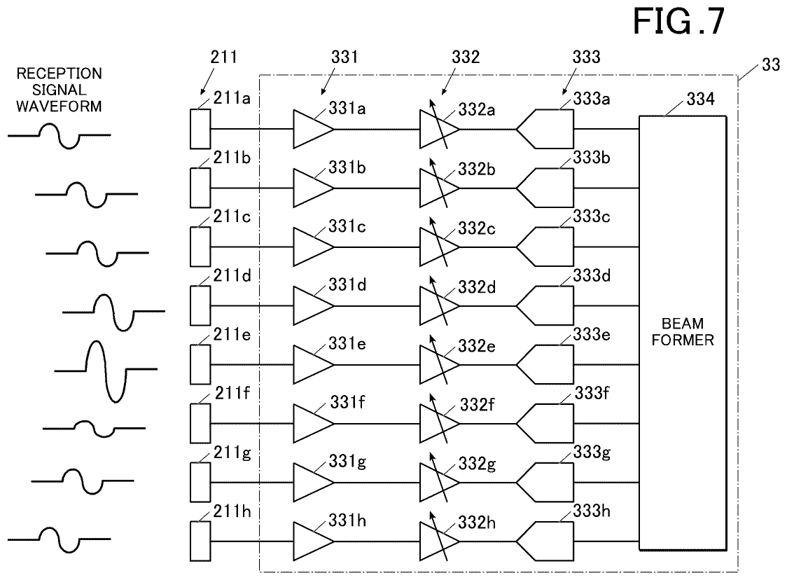

A second embodiment of the present invention will be described with reference to FIG. 6. FIG. 6 is a flowchart illustrating second image display processing.

The first embodiment includes a configuration in which the ultrasound image data without saturation is generated from the ultrasound image data with saturation (color Doppler image data) using the estimation data. The present embodiment is configured to, in a case where the ultrasound image data with saturation (color Doppler image data) is acquired, reduce the gain of the receiver 13 to generate the ultrasound image data without saturation.

In the present embodiment, the ultrasound diagnostic apparatus 100 is used as an apparatus configuration. It is assumed that a learning program and a second image display program for executing a second image display process to be described later are stored in the ROM of the controller 18.

Next, the operation of the ultrasound diagnostic apparatus 100 according to the present embodiment will be described with reference to FIG. 6. The second image display processing executed by the ultrasound diagnostic apparatus 100 will be described with reference to FIG. 6. The second image display processing is processing in which the ultrasound image data of the subject such as the patient for diagnosis is acquired, and in a case where saturation is present, the gain is reduced to generate and display the ultrasound image data without saturation. In addition, since the description of the learning processing is the same as that of the first embodiment, the description will be omitted.

It is assumed that a gain initial value which is an initial value of each gain value for the color Doppler image data of the variable gain amplifier 132 is stored in the storage 19 in advance.

In the ultrasound diagnostic apparatus 100, after the learning processing, for example, an instruction to execute the second image display processing is input from the user via the operation inputter 11. In response to the execution instruction, the controller 18 executes a second image display process in accordance with a second image display program stored in the ROM.

First, the controller 18 reads and acquires the gain initial value from the storage 19 (step S31). The controller 18 sets each gain value of the variable gain amplifier 132 in accordance with the gain initial value in step S31 or the gain value decreased in step S36 (step S32). That is, when step S32 is executed for the first time, the gain initial value is set. At the time of performing step S32 thereafter, the lowered gain value of step S36 is set.

The controller 18 acquires the ultrasound image of the subject as the target to be diagnosed by the control of the transmitter 12 to the display controller 16 including the control of the gain value set in step S32 (step S33). In step S33, the variable gain amplifiers 132a to 132h are controlled with the gain values set in step S32. The color Doppler image data and the B-mode image data corresponding to the set gain value are generated. Steps S34, S35, and S36 are the same as steps S22, S23, and S24 of the first image display process of FIG. 5, respectively.

In a case where the saturation is present (step S36; YES), the controller 18 decreases the set gain value corresponding to the region in which the saturation is determined to be present by a predetermined amount (step S37). The processing proceeds to step S32. If there is no saturation (step S36; NO), the controller 18 displays the ultrasound image data acquired in the immediately preceding step S33 on the display 17 (step S38). In step S38, the color Doppler image data and the B-mode image data acquired in the immediately preceding step S33 are superimposed and displayed. The second image display processing ends.

As described above, according to the present embodiment, the controller 18 determines whether or not the ultrasound image data based on the reception signal of the ultrasound probe 2 is saturated by the determination data of the learned data. When saturation is present, the controller 18 decreases the gain of the variable gain amplifier 132, which amplifies the reception signal at any degree, so as not to saturate the reception signal, and causes the ultrasound image data to be generated. Therefore, even in the case of saturation due to a strong echo, it is possible to obtain an ultrasound image (color Doppler image) with good image quality without saturation. In addition, since the ultrasound image data without saturation is not generated by image processing, it is possible to reduce the image processing burden.

The controller 18 determines, with the determination data, whether the ultrasound image data based on the reception signals of the ultrasound probe 2 includes the saturated region. When there is a region with saturation, the controller 18 generates the ultrasound image data by reducing the gain of the region of the variable gain amplifier 132 for amplifying the reception signal at any degree so as not to be saturated. For this reason, even in the case of saturation due to a strong echo, it is possible to obtain the ultrasound image with good image quality without saturation, and it is possible to reduce the processing load of the gain setting.

In the above description, an example in which the ROM of the controller 18 is used as a computer-readable medium of the program according to the present invention is disclosed, but the present invention is not limited to this example. As other computer-readable media, a nonvolatile memory, such as a flash memory, and a portable recording medium, such as a CD-ROM, can be applied. Furthermore, a carrier wave is also applied as a medium for providing data of a program according to the present invention via a communication line.

Note that the description in the above embodiment is an example of the ultrasound diagnostic apparatus, the information processing apparatus, the ultrasound image generating method, the ultrasound image learning method, and the program according to the present invention, and the present invention is not limited to this.

In each of the above embodiments, the ultrasound image data serving as image data is subjected to machine learning to generate learned data. Furthermore, the first embodiment has a configuration in which estimated image data without saturation is generated from the ultrasound image data with saturation serving as image data. However, the present invention is not limited to this configuration. As the image data, sound ray data or intermediate data generated between sound ray data generation and image data generation may be used.

Furthermore, in each of the above-described embodiments, the ultrasound diagnostic apparatus 100 as the information processing apparatus is configured to perform machine learning by using the ultrasound image data with/without saturation. However, the present invention is not limited to this configuration. For example, a server as the information processing apparatus may be provided on a communication network connected to the ultrasound diagnostic apparatus 100. The server acquires the ultrasound image data with/without saturation generated by the ultrasound diagnostic apparatus 100 and generates the estimation data by machine learning. The server transmits the estimation data to the ultrasound diagnostic apparatus 100 for storage.

The above-described first embodiment is configured to perform machine learning of the color Doppler image data with/without saturation in the color Doppler mode. Whether or not the acquired color Doppler image data is saturated is determined. In the case of presence of saturation, the color Doppler image data without saturation is estimated and generated. The estimated color Doppler image data without saturation is displayed with the B-mode image data superimposed thereon. However, the present invention is not limited to this configuration. For example, the color Doppler image data and the B-mode image data with/without saturation may be machine-learned. The presence or absence of saturation (of the region) in the acquired color Doppler image data is determined. In the case of presence of saturation, the color Doppler image data without saturation is estimated and generated. The presence or absence of saturation (of the region) in the acquired B-mode image data is determined. In the case of presence of saturation, the B-mode image data without saturation is estimated and generated. The estimated color Doppler image data without saturation and the B-mode image data without saturation are superimposed and displayed.

Furthermore, for example, superimposed image data of the color Doppler image data and B-mode image data with/without saturation may be subjected to machine learning. The presence or absence of saturation (of a region) in the acquired superimposed image data is determined. In the case of saturation, superimposed image data without saturation is estimated, generated, and displayed. Furthermore, ultrasound image data with/without saturation in another image mode such as the B mode other than the color Doppler mode may be subjected to machine learning. The presence or absence of saturation (of the region) in the acquired ultrasound image data is determined. In the case of saturation, ultrasound image data without saturation is estimated, generated, and displayed. The above-described determination is also applicable to the second embodiment.

In addition, the detailed configuration and the detailed operation of the ultrasound diagnostic apparatus 100 in the present embodiment described above can be appropriately modified without departing from the spirit and scope of the present invention.

Although embodiments of the present invention have been described and shown in detail, the disclosed embodiments are made for purposes of illustration and example only and not limitation. The scope of the present invention should be interpreted by terms of the appended claims.

The entire disclosure of Japanese Patent Application No. 2023-200491, filed on Nov. 28, 2023, including description, claims, drawings and abstract is incorporated herein by reference.

What is claimed is:

1. An ultrasound diagnostic apparatus comprising:
a hardware processor,
wherein the hardware processor generates image data without saturation from image data with saturation using learned data of a model that has undergone machine learning using the image data with saturation based on a reception signal of an ultrasound probe and the image data without saturation.

2. The ultrasound diagnostic apparatus according to claim 1, wherein the hardware processor determines, based on the learned data, whether the image data based on the reception signal of a transducer of the ultrasound probe is saturated, and when it is determined to be saturated, the hardware processor generates the image data by reducing a gain of a variable gain amplifier that amplifies the reception signal at any degree so as not to be saturated.

3. The ultrasound diagnostic apparatus according to claim 2, wherein the hardware processor determines, based on the learned data, whether the image data based on the reception signal of the ultrasound probe includes a saturated region, and when it is determined that there is the saturated region, the hardware processor generates the image data by decreasing, so as not to saturate, the gain of the region of the variable gain amplifier that amplifies the reception signal at any degree.

4. The ultrasound diagnostic apparatus according to claim 1, wherein the hardware processor determines, based on the learned data, whether the image data based on the reception signal of the ultrasound probe is saturated, and when it is determined to be saturated, the hardware processor estimates and generates, based on the learned data, the image data of a non-saturated region from the image data.

5. The ultrasound diagnostic apparatus according to claim 4, wherein the hardware processor determines, based on the learned data, whether the image data based on the reception signal of the ultrasound probe includes the saturated region, and when it is determined that there is the saturated region, estimates and generates, with the learned data, the image data of the non-saturated region from the image data of the region, and generates the image data including the non-saturated region.

6. An ultrasound image generating method comprising:
generating image data without saturation from image data with saturation using learned data of a model that has undergone machine learning using the image data with saturation based on a reception signal of an ultrasound probe and the image data without saturation.

7. A non-transitory computer-readable recording medium storing a program that causes a computer to:
as a controller, generate image data without saturation from image data with saturation using learned data of a model that has undergone machine learning using the image data with saturation based on a reception signal of an ultrasound probe and the image data without saturation.
